(19) United States
(12) Patent Application Publication
(10) Pub. No.: US 2016/0074409 A1
(43) Pub. Date: Mar. 17, 2016

(54) Treatment of Skeletal-Related Disorders

(71) Applicants: Wayne Rothbaum, New York, NY (US); Glenn Begley, New York, NY (US)

(72) Inventors: Wayne Rothbaum, New York, NY (US); Glenn Begley, New York, NY (US)

(21) Appl. No.: 14/795,384

(22) Filed: Jul. 9, 2015

Related U.S. Application Data
(63) Continuation of application No. 14/186,902, filed on Feb. 21, 2014, now abandoned.
(60) Provisional application No. 61/767,645, filed on Feb. 21, 2013.

(51) Int. Cl. A61K 31/53; A61K 31/519; A61N 5/00; A61K 31/505

(52) U.S. Cl. CPC A61K 31/53; A61K 31/505; A61K 31/519; A61N 5/00

(57) ABSTRACT

The invention relates to the prevention and/or treatment of skeletal related disorders using heteroaryl compounds.

### TECHNICAL FIELD OF THE INVENTION

The present invention relates to methods of using heteroaryl compounds in the prevention and/or treatment of skeletal-related disorders.

### BACKGROUND OF THE INVENTION

The osteoclast is a terminally differentiated cell derived from monocytic/macrophage lineage which resorbs bone as part of the normal process of skeletal modeling and remodeling. In contrast to precursor cells, only fully differentiated mature osteoclasts are able to resorb bone. Increased osteoclastic bone resorption has been linked to the pathogenesis of several skeletal disorders, most notably post-menopausal osteoporosis and skeletal metastases.

Osteoclast precursor cells possess a receptor, receptor activator of NF-κB (RANK) that recognizes a ligand (RANKL) which leads to osteoclast differentiation. The RANKL receptor is a member of the tumor necrosis factor (TNF) family and has previously been shown to be an activator of NF-κB and is a specific inducer of osteoclastogenesis. It has been shown that RANKL is a key regulator of osteoclastogenesis and that the PI3 kinase complex is associated with the RANKL receptor. It has been reported that PI3 kinase is involved with ruffled border formation in osteoclasts and inhibition of PI3 kinase will affect osteoclast attachment and spreading leading to subsequent osteopenia.

It has also been found that Bruton's Tyrosine Kinase (BTK) and intermediates in the BTK pathway are critical intermediates in the cytoskeletal rearrangement pathway leading to osteoclast activation. For example, elevated cytokine production restores bone resorption by human BTK-deficient osteoclasts (Danks (2011) Journal of Bone and Mineral Research, 26, 182-192). Further, tyrosine kinases BTK and Tec regulate osteoclast differentiation by linking RANK and ITAM signals (Shinohara (2008) Cell 132, 794-806). Mice deficient in BTK exhibit osteopenia and this osteopenia can be reversed upon the addition of multiple copies of the BTK gene in transgenic mice (see EP 1373554). Accordingly, modulators of BTK activity and Tec kinase activity are useful in affecting osteoclast activation and bone resorption.

### SUMMARY OF THE INVENTION

One aspect of the invention provides a method of preventing and/or treating a skeletal-related event in a human subject with bone metastases from solid tumors or multiple myeloma comprising administering to the human subject an amount of a compound of Formula I-a or I-b:

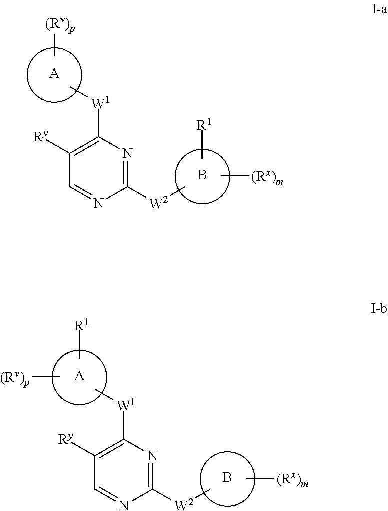

or a pharmaceutically acceptable salt thereof, effective to inhibit the activity of BTK in the human subject, wherein:
a. Ring A is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms Independently selected from nitrogen, oxygen, or sulfur;
b. Ring B is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
c. R¹ is a warhead group;
d. R^{V} is hydrogen, halogen, —CN, —CF₃, C₁₋₄ aliphatic, C₁₋₄ haloaliphatic, —OR, —C(O)R, or —C(O)N(R)₂;
e. each R group is independently hydrogen or an optionally substituted group selected from C₁₋₆ aliphatic, phenyl, a 4-7-membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
f. W¹ and W² are each independently a covalent bond or a bivalent C₁₋₃ alkylene chain wherein one methylene unit of W¹ or W² is optionally replaced by —NR₂—, —N(R²)C(O)—, —C(O)N(R²)—, N(R²)SO₂—, SO₂N(R²)—, —O—, —C(O)—, —OC(O)—, —C(O)O—, —S—, —SO— or —SO₂—;
g. R² is hydrogen, optionally substituted C₁₋₆aliphatic, or —C(O)R, or;
h. R² and a substituent on Ring A are taken together with their intervening atoms to form a 4-6 membered partially unsaturated or aromatic fused ring; or
i. R² and R^{y} are taken together with their intervening atoms to form a 4-6 membered saturated, partially unsaturated, or aromatic fused ring; m and p are independently 0-4; and
j. R^{x} and R^{y} are independently selected from —R, halogen, —OR, —O(CH₂)_{q}OR, —CN, —NO, —SO₂R, —SO₂N(R)₂, SOR, —C(O)R, —CO₂R, —C(O)N(R)₂, —NRC(O)R, —NRC(O)N(R)₂, —NRSO₂R, or —N(R)₂, wherein q is 1-4; or;
k. R^{x} and R¹ when concurrently present on Ring B are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, CN, or C₁₋₆ aliphatic; or
l. R^{v} and R¹ when concurrently present on Ring A are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, CN, or C₁₋₆ aliphatic.

Another aspect of the invention provides a method of preventing and/or treating a skeletal-related event in a human subject with bone metastases from solid tumors or multiple myeloma comprising administering to the human subject an amount of a compound of Formula II

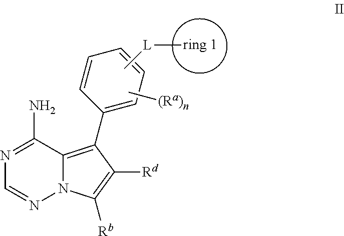

or a pharmaceutically acceptable salt thereof, effective to inhibit the activity of Bruton's tyrosine kinase (Btk) in the human subject, wherein:
a. ring1 represents (1) a C₅₋₇ carbocyclic ring or (2) a 5-10 membered heterocyclic or heteroaromatic ring, any of which is optionally substituted with 1-5 substituent(s) selected from the group consisting of halogen, a C₁₋₄alkyl, CF₃, nitrile, CONH₂, and OR^{e-103};
b. R^{a} represents halogen, a C₁₋₄alkyl, or a C₃₋₄alkoxy;
c. L represents —O—, —SO—, —SO₂—, —NH—, or —C(O)—;
d. R^{b} represents (1) a C₁₋₄alkyl substituted with OR^{s-103}, (2) C₂₋₄alkenyl, or (3) ring2 optionally substituted with one or more —K—R^{c};
e. ring2 represents (1) a C₄₋₇carbocyclic ring or (2) a 4-7 membered heterocyclic or heteroaromatic ring, any atom of which is optionally substituted with one or more oxo group; K represents bond, a C₁₋₄alkylene, —C(O)CH₂—, —C(O)CH₂CH₂—, —C(O)O—, CH₂C(O)—, —CH₂C(O)O—, —C(O)—, —CH₂O—, —CH₂CH₂O—, —O—, —OCH₂—, —OCH₂C(O)— or —SO₂—, wherein the left bond binds to ring2;
f. R^{c} represents (1) hydrogen, (2) NR^{c-101}R^{c-102}, (3) a C₁₋₄alkyl optionally substituted with NR^{c-101}R^{c-102}, (4) a C₂₋₄alkenyl optionally substituted with NR^{c-101}R^{c-102}, (5) CF₃, (6) nitrile, (7) halogen, or (8) a cyclic ring optionally substituted with 1-5 substituent(s) selected from the group consisting of halogen, a C₁₋₄alkyl, a C₁₋₄alkoxy, CF₃, nitrile and oxo, wherein the cyclic ring is selected from the group consisting of morpholine, pyrrolidine, benzene, piperazine, tetrahydropyran, piperidine, tetrahydrofuran, oxazole, thiazole, pyrazole and oxadiazole;
g. R^{d} represents (1) halogen, (2) CONR^{d-101}R^{d-102}, (3) CO₂R^{d-103}, (4) ring3, (5) a C₁₋₄CONR^{d-101}R^{d-102}, CO₂R^{d-103}, COR^{d-103}, OR^{d-103}, SOR^{d-103} and SO₂R^{d-102}, or (6) a C₂₋₄alkenyl which is substituted with 1-5 substituent(s) selected from ring4, nitrile, NR^{d-103}R^{d-102}, CONR^{d-101}R^{d-102}, CO₂R^{d-103}, COR^{d-103}, OR^{d-103}, SOR^{d-103} and SO₂R^{d-103};
h. R^{c-101} and R^{c-102} each independently represent (1) hydrogen, (2) a C₁₋₄alkyl, (3) COR^{c-103}, (4) CONR^{c-103}R^{c-104} or (5) SO₂R^{c-103}, wherein R^{c-103} and R^{c-104} each independently represent hydrogen or a C₁₋₄alkyl;
i. R^{d-101}, R^{d-102} and R^{d-103} each independently represent (1) hydrogen, (2) COR^{e-103}, (3) NR^{e-101}R^{e-102}, (4) ring5, or (6) a C₁₋₄-alkyl optionally substituted with CO₂R^{e-103}, OR^{e-103}, or NR^{e-103}R^{e-102}; R^{e-101},
j. R^{e-102} and R^{e-103} each independently represent hydrogen or a C₁₋₄alkyl;
k. ring3, ring4 and ring5 each independently represent a 4-7 membered heterocyclic or heteroaromatic ring optionally substituted with 1-5 substituent(s) selected from the group consisting of halogen, oxo, a C₁₋₄alkyl, a C₁₋₃alkoxy, CF₃, CONR^{e-101}R^{e-102}, CO₂R^{e-103}, SOR^{e-103}, SO₂R^{e-103} and nitrile;
l. n represents 0, or an integer of 1-4, wherein when n is more than 1, and
m. each R^{a} may be same or different.

Yet another aspect of the invention provides a method of preventing and/or treating a skeletal-related event in a human subject with bone metastases from solid tumors or multiple myeloma comprising administering to the human subject an amount of a compound of Formula III

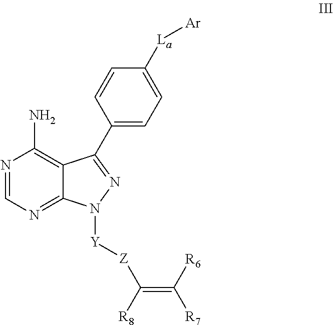

or a pharmaceutically acceptable salt thereof, effective to inhibit the activity of Bruton's tyrosine kinase (Btk) in the human subject, wherein:
a. La is O or S;
b. Ar is an unsubstituted phenyl;
c. Y is a 4-, 5-, 6-, or 7-membered cycloalkyl ring, or Y is azetidinyl, pyrrolidinyl, piperidinyl, or azepanyl;
d. Z is C(═O), OC(═O), NHC(═O), S(═O)ₓ, or NHS(═O)ₓ, where x is 2;
e. R₈ is H;
f. R₇ is H, unsubstituted C₁-C₄alkyl, C₁-C₆alkoxyalkyl, C₁-C₈alkylaminoalkyl, or C₁-C₄alkyl(phenyl); or
g. R₇ and R₈ taken together form a bond; and
h. R₆ is H, unsubstituted C₁-C₄alkyl, C₁-C₆alkoxyalkyl, C₁-C₈alkylaminoalkyl, or C₁-C₄alkyl(phenyl).

In a particular embodiment of Formula I-a and I-b, the compound is

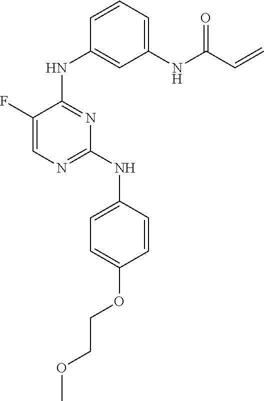 [SMILES: C=CC(=O)Nc1cccc(Nc2nc(Nc3ccc(OCCOC)cc3)ncc2F)c1]

or a pharmaceutically acceptable salt thereof.

In a particular embodiment of Formula II, the compound is

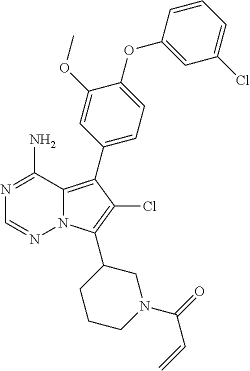 [SMILES: C=CC(=O)N1CCCC(c2c(Cl)c(-c3ccc(Oc4cccc(Cl)c4)c(OC)c3)c3c(N)ncnn23)C1]

or a pharmaceutically acceptable salt thereof.

In a particular embodiment of Formula III, the compound is

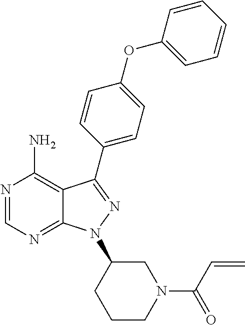 [SMILES: C=CC(=O)N1CCC[C@@H](n2nc(-c3ccc(Oc4ccccc4)cc3)c3c(N)ncnc32)C1]

or a pharmaceutically acceptable salt thereof.

Yet another aspect of the invention provides a method of preventing and/or treating a skeletal-related event in a human subject with bone metastases from solid tumors or multiple myeloma comprising administering to the human subject an amount of a compound of Formula IV with the following structure:

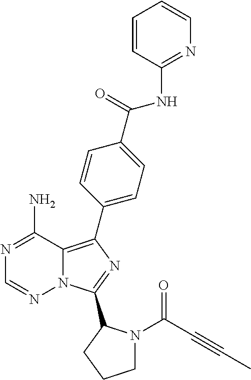

or a pharmaceutically acceptable salt thereof.

### BRIEF DESCRIPTION OF DRAWINGS

FIG. 1: Femoral and tibial X-ray densitometry results in metastatic bone cancer model. G1: vehicle BID PO (N=6), G2: 0.04 mg/kg zoledronate QWK IV (N=6), G5: 90 mg/kg Compound of Formula IV BID PO (N=6), and G6: No implantation (N=3). Results are comparative between left (experimental) and right (control) limbs.

### DETAILED DESCRIPTION

Compounds included in the methods of the invention comprise those described generally above and the species disclosed herein. As used herein, the following definitions shall apply unless otherwise indicated. For purposes of this invention, the chemical elements are identified in accordance with the Periodic Table of the Elements, CAS version. Handbook of Chemistry and Physics, 75^{th} Ed. General principles of organic chemistry are described in, for example, March's Advanced Organic Chemistry, (5^{th} Ed., Ed.: Smith, M. B. and March, J., John Wiley & Sons, New York: 2001), the entire contents of which are hereby incorporated by reference.

The term “aliphatic” or “aliphatic group”, as used herein, means a straight-chain (I.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cydoaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1.2 aliphatic carbon atoms. In some embodiments, “cycloaliphatic” (or “carbocycle” or “cycloalkyl”) refers to a monocyclic C₃₋₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. For example, a C₂₋₄alkenyl would include, for example, straight and branched chain C₂₋₄ alkenyl groups, such as ethenyl, 1-propenyl, 2-propenyl, 1-butenyl, 2-butenyl, and 3-butenyl. Carbocyclic rings include, but are not limited to, cyclobutane, cyclopentane, cyclohexane, cycloheptane, cyclobutene, cyclopentene, cyclohexene, cycloheptene, cyclobutadiene, cyclopentadiene, cyclohexadiene and cycloheptadiene.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “alkoxy” refers to straight or branched alkyl groups containing a oxygen atom that is not bound to hydrogen. Examples include, but are not limited to, methoxy, ethoxy, propoxy, isopropoxy, butoxy, isobutoxy, sec-butoxy, and tert-butoxy

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

The term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. In particular, a C₁₋₄ alkylene would include, for example, methylene, ethylene, trimethylene, tetramethylene and isomers thereof. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “cyclopropylenyl” refers to a bivalent cyclopropyl group of the following structure:

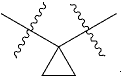 [SMILES: *C1(*)CC1]

The term “halogen” refers to F, Cl, Br, or I.

The term “aryl” used alone or as part of a larger moiety (such as in “aralkyl”, “aralkoxy”, or “aryloxyalkyl”) refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In selected embodiments of the invention, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group such as indanyl, phthalimidyl, naphthimidyl, phenanthridinyl, or tetrahydronaphthyl and the like in which an aromatic ring is fused to one or more non-aromatic rings.

The terms “heteroaryl” and “heteroar-”, used alone or as part of a larger moiety (e.g., “heteroaralkyl”, or “heteroaralkoxy”) refer to groups having 5 to 10 ring atoms (preferably 5, 6, or 9 ring atoms) having 6, 10, or 14 π-electrons shared in a cyclic array; and having from one to five heteroatoms in addition to the carbon atoms. The term “heteroatom” refers to nitrogen, oxygen, or sulfur atoms, and includes any oxidized form of nitrogen or sulfur, and any quaternized form of nitrogen. Heteroaryl groups include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Examples include, but are not limited to, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one A heteroaryl group may be monocyclic or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having one or more (preferably one to four) heteroatoms in addition to the carbon atoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or *NR (as in N-substituted pyrrolidinyl).

A heterocyclic ring can be attached to its pendant group at any heteroatom or carbon atom that results in a stable structure and any of the ring atoms may be optionally substituted. Examples of such saturated or partially unsaturated heterocyclic radicals include, but are not limited to, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocycyl”, “heterocyclyl ring”, “heterocyclic group”, “heterocyclic moiety”, and “heterocyclic radical” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be monocyclic or bicyclic. The term “heterocyclylalkyl” refers to an alkyl group substituted by a heterocyclyl, wherein the alkyl and heterocyclyl portions independently are optionally substituted.

The term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group independently include, but are not limited to, halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄—CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; N(R^{o})C(S)R^{o}; —(CH₃)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; C(S)Rₒ; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SIR^{o}₃; —(C₃₋₄ straight or branched alkylene)O—N(R^{o})₂; or —(C₁₋₄ straight or branched alkylene)C(O)O—N(R^{o})₂, wherein each R^{o} may be substituted as defined below and is independently hydrogen, C₂₋₆ aliphatic, —CH₂Ph, —O(CH₂)₀₋₁Ph, —CH₂—(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), to form a 3-12-membered saturated, partially unsaturated, or aryl monocyclic or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms) include, but are not limited to, independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R*, -(haloR*), —OH, —OR*, —O(haloR*), —CN, —C(O)OH, —C(O)OR*, —NH₂, —NHR*, —NR*₂, or —NO₂, wherein each R* is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₃₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cydopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In some embodiments, the R³ group of formula I-a and I-b comprises one or more deuterium atoms.

Particular compounds of Formula I include, but are not limited to, N-(3-(5-methyl-2-(phenylamino)pyrimidin-4-ylamino)phenyl)acrylamide; N-(3-(4-(m-tolylamino)pyrimidin-2-ylamino)phenyl)acrylamide; N-(3-(5-methy-4-(m-tolyamino)pyrimdin-2-ylamino)phenyl)acrylamide; N-(3-(5-fluoro-4-(m-tolylamino)pyrimidin-2-ylamino)phenyl)acrylamide; (E)-4-(dimethylamino)-N-(3-(5-fluoro-4-(m-tolylamino)pyrimidin-2-ylamino)-phenyl)but-2-enamide; N-(3-(5-methyl-4-(phenylamino)pyrimidin-2-ylamino)phenyl)acrylamide; N-(4-methyl-3-(5-methyl-4-(m-tolylamino)pyrimidin-2-ylamino)phenyl)acrylamide; N-(3-(4-(3-bromophenylamino)-5-methylpyrimidin-2-ylamino)phenyl)acrylamide; 3-(4-(2-cyclopropylsulfonyl)-1,2,3,4-tetrahydroisoquinolin-6-ylamino)-5-methylpyrimidin-2-ylamino)benzenesulfonamide; 3-(4-(2-(2-chloroacetyl)-1,2,3,4-tetrahydroisoquinolin-6-ylamino)-5-methylpyrimidin-2-ylamino)benzenesulfonamide; N-(3-(5-methyl-4-(4-phenoxyphenylamino)pyrimidin-2-ylamino)phenyl)acrylamide; N-(3-(5-methyl-2-(3-sulfamoylphenylamino)pyrimidin-4-ylamino)phenyl)acrylamide; N-(3-(methyl(5-methyl-2-(phenylamino)pyrimidin-4-yl)amino)phenyl)acrylamide; N-(3-(5-methyl-2-(3-(prop-2-ynyloxy)phenylamino)pyrimidin-4-ylamino)phenyl)acrylamide; (E)-4-(dimethylamino)-N-(3-(5-methyl-2-(phenylamino)pyrimidin-4-ylamino)phenyl)but-2-enamide; N-(4-(5-methyl-2-(phenylamino)pyrimidin-4-ylamino)phenyl)acrylamide; N-(4-methyl-3-(4-(pyridin-3-yl)pyrimidin-2-ylamino)phenyl)acrylamide; N-(3-(5-fluoro-2-(4-(2-methoxyethoxy)phenylamino)pyrimidin-4-ylamino)phenyl)acrylamide; N-(3-(2-(3-chloro-4-(pyridin-2-ylmethoxy)phenylamino)-5-fluoropyrimidin-4-ylamino)phenyl)acrylamide; N-(3-(5-fluoro-2-(4-(2-(2-oxopyrrolidin-1-yl)ethoxy)phenylamino)pyrimidin-4-ylamino)phenyl)acrylamide; N-(3-(S-fluoro-2-(4-(1-hydroxy-2-methylpropan-2-yloxy)phenylamino)pyrimidin-4-ylamino)phenyl)acrylamide; N-(3-(5-fluoro-2-(6-isopropoxypyridin-3-ylamino)pyrimidin-4-ylamino)phenyl)acrylamide; N-(3-(S-fluoro-2-(2-oxoindolin-5-ylamino)pyrimidin-4-ylamino)phenyl)acrylamide; N-(2-chloro-5-(5-fluoro-2-(4-(2-methoxyethoxy)phenylamino)pyrimidin-4-ylamino)phenyl)acrylamide; N-(2-chloro-5-(5-fluoro-2-(6-isopropoxypyridin-3-ylamino)pyrimidin-4-ylamino)phenyl)acrylamide; N-(2-fluoro-5-(5-fluoro-2-(4-(2-methoxyethoxy)phenylamino)pyrimidin-4-ylamino)phenyl)acrylamide; N-(3-(5-fluoro-2-(4-((1-methylpiperidin-4-yl)methoxy)phenylamino)pyrimdin-4-ylamino)phenyl)acrylamide; N-(3-(5-fluoro-2-(6-((2-methoxyethyl)(methyl)amino)pyridin-3-ylamino)pyrimidin-4-ylamino)phenyl)acrylamide; 1-(6-(5-fluoro-2-(6-methoxypyridin-3-ylamino)pyrimidin-4-ylamino)-2H-benzo[b][1,4]oxazin-4(3H)-yl)prop-2-en-1-one; 1-(6-(5-fluoro-2-(4-(2-methoxyethoxy)phenylamino)pyrimidin-4-ylamino)-2H-benzo[b][1,4]oxazin-4(3H)-yl)prop-2-en-1-one; and 1-(6-(5-fluoro-2-(6-methoxypyridin-3-ylamino)pyrimidin-4-ylamino)indolin-1-yl)prop-2-en-1-one.

Particular compounds of Formula II include, but are not limited to, 5-(4-(3-chlorophenoxy)-3-methoxyphenyl)-7-(3-methoxyphenyl)-6-(3(pyrrolidin-1-yl)propyl)pyrrolo[2,1-f][1,2,4]triazin-4-amine; 6-(3-(dimethylamino)propyl)-7-(3-methoxyphenyl)-5-(4-phenoxyphenyl)pyrrolo[2,1-f][1,2,4]triazin-4-amine; 7-(3-methoxyphenyl)-5-(4-phenoxyphenyl)-6-(3-(pyrrolidin-1-yl)propyl)pyrrolo[2,1-f][1,2,4]triazin-4-amine; 5-(4-(3,4-dichlorophenoxy)-3-methoxyphenyl)-7-(3-methoxyphenyl)-6-(3-(pyrrolidin-1-yl)propyl)pyrrolo[2,1-f][1,2,4]trazin-4-amine; 5-[4-(3-chlorophenoxy)phenyl]-7-(3-methoxyphenyl)-6-(4-morpholinylmethyl)-pyrrolo[2,1-f][1,2,4]triazin-4-amine; 5-[4-(3-chlorophenoxy)phenyl]-7-cyclopentyl-6-(4-morpholinylmethyl)pyrrolo[2,1-f][1,2,4]triazin-4-amine, [0304]; 1-(4-(4-amino-5-(4-(3,4-dichlorophenoxy)-3-methoxyphenyl)-6-(3-(pyrrolidin-1-yl)propyl)pyrrolo[2,1-f][1,2,4]triazin-7-yl)piperidin-1-yl)-2-(diethylamino)ethanone; 1-(4-(4-amino-5-(4-(3-chlorophenoxy)-3-methoxyphenyl)-6-(3-(pyrrolidin-1-yl)propyl)pyrrolo[2,1-f][1,2,4]triazin-7-yl)piperidin-1-yl)-2-(diethylamino)ethanone; 7-cyclopentyl-5-(4-(3,4-dichlorophenoxy)-3-methoxyphenyl)-6-(2-(pyridin-3-yl)ethyl)pyrrolo[2,1-f][1,2,4]triazin-4-amine; 7-(3-methoxyphenyl)-5-(4-phenoxyphenyl)-6-[2 (3-pyridinyl)ethyl]pyrrolo[2,1-f][1,2,4]triazin-4-amine; 5-[4-(3,4-dichlorophenoxy)phenyl]-7-(3-methoxyphenyl)-6-[2-(3-pyridinyl)ethyl]pyrrolo[2,1-f][1,2,4]triazin-4-amine; 7-(3-methoxyphenyl)-5-(4-phenoxyphenyl)-6-(piperidin-4-yl)pyrrolo[2,1-f][1,2,4]triazin-4-amine; 7-cyclopentyl-5-(4-(3,4-dichlorophenoxy)-3-methoxyphenyl)-6-(piperidin-4-yl)pyrrolo[2,1-f][1,2,4]triazin-4-amine; 5-(4-(3-chlorophenoxy)-3-methoxyphenyl)-7-cyclopentyl-6-(piperidin-4-yl)pyrrolo[2,1-f][1,2,4]triazin-4-amine; 3-(4-(4-amino-7-cyclopentyl-6-(piperidin-4-yl)pyrrolo[2,1-f][1,2,4]triazin-5-yl)-2-methoxyphenoxy)benzonitrile; 5-[4-(3,4-dichlorophenoxy)-3-methoxy-phenyl]-7-(1-methylsulfonyl-4-piperidyl)-6-(4-piperidyl)pyrrolo[2,1-f][1,2,4]triazin-4-amine; 5-(4-(3-chlorophenoxy)-3-methoxyphenyl)-7-(1-(methylsulfonyl)piperidin-4yl)-6-(piperidin-4-yl)pyrrolo[2,1-f][1,2,4]triazin-4-amine; (3-{4-amino-5-[4-(3-chlorophenoxy)-3-methoxyphenyl]-6-(4-pyridinyl)pyrrolo[2,1-f][1,2,4]triazin-7-yl}phenyl)(4-methyl-1-piperazinyl)methanone; 7-cycloheptyl-5-[4-(3,4-dichlorophenoxy)-3-methoxyphenyl]-6-(4 piperidinyl)pyrrolo[2,1-f][1,2,4]triazin-4-amine; 5-[4-(3-chlorophenoxy)-3-methoxyphenyl]7-cycloheptyl-6-(4-piperidinyl)pyrrolo[2,1-f][1,2,4]trazin-4 amine; 5-[4-(3,4-dichlorophenoxy)-3-methoxyphenyl]-7-(3-methoxyphenyl)-6-(4 piperidinyl)pyrrolo[2,1-f][1,2,4]triazin-4-amine; 5-[4-(3-chlorophenoxy)-3-methoxyphenyl]-7-(3-methoxyphenyl)-6-(4-piperidinyl)pyrrolo[2,1-f][1,2,4]triazin-4-amine; N-(4-{4-amino-S-[4-(3-chlorophenoxy)-3-methoxyphenyl]-6-(4-piperidinyl)pyrrolo[2,1-f][1,2,4]triazin-7-yl}phenyl)-N-methylmethanesulfonamide; 5-[4-(3-chlorophenoxy)-3-methoxyphenyl]-7-(4-methoxyphenyl)-6-(4-piperdinyl)pyrrolo[2,1-f][1,2,4]triazin-4-amine; N-(4-{4-amino-5-[4-(3,4-dichlorophenoxy)-3-methoxyphenyl]-6-(4-piperidinyl)pyrrolo[2,1-f][1,2,4]triazin-7-yl}phenyl)-N-methylmethanesulfonamide; 3-{-4-[4-amino-7-(4-methoxyphenyl)-6-(4-piperidinyl)pyrrolo[2,1-f][1,2,4]-triazin-5-yl]-2-methoxyphenoxy}benzonitrile; 6-chloro-5-(4-(3-chlorophenoxy)-3-methoxyphenyl)-7-(1-(methylsulfonyl)piperidin-4-yl)pyrrolo[2,1-f][1,2,4]triazin-4-amine; 1-(3-(4-amino-6-chloro-5-(4-(3-chlorophenoxy)-3-methoxyphenyl)pyrrolo[2,1-f][1,2,4]triazin-7-yl)piperidin-yl)prop-2-en-1-one; 1-(3-(4-amino-6-chloro-5-(4-(3,4-dichlorophenoxy)-3-methoxyphenyl)pyrrolo-[2,1-f][1,2,4]triazin-7-yl)piperidin-1-yl)prop-2-en-1-one; 5-(4-(3-chlorophenoxy)-3-methoxyphenyl)-7-cyclopentyl-6-(pyrrolidin-3-yl)-pyrrolo[2,1-f][1,2,4]triazin-4-amine; 5-[4-(3-chlorophenoxy)phenyl]-7-cyclopentyl-6-(2-ethoxyethyl)pyrrolo[2,1-f][1,2,4]triazin-4-amine; 3-(4-amino-5-[4-(3,4-dichlorophenoxy)-3-methoxyphenyl]-7-(3-methoxyphenyl)pyrrolo[2,1-f][1,2,4]triazin-6-yl)propanoic acid; {4-amino-7-cyclopentyl-5[4-(3,4-dichlorophenoxy)-3-methoxyphenyl]pyrrolo-[2,1-f][1,2,4]triazin-6-yl}acetic acid; 2-(4-amino-7-cyclopentyl-5-[4-(3,4-dichlorophenoxy)-3-methoxyphenyl]pyrrolo[2,1-f][1,2,4]triazin-6-yl)ethanol; methyl 4-amino-7-(3-methoxyphenyl)-5-(4-phenoxyphenyl)pyrrolo[2,1-f][1,2,4]triazine-6-carboxylate; 4-amino-7-(3-methoxyphenyl)-5-(4-phenoxyphenyl)pyrrolo[2,1-f][1,2,4]triazine-6-carboxylic acid; ethyl 3-{7-(1-acryloyl-3-piperidinyl)-4-amino-5-[4-(3-chlorophenoxy)-3-methoxyphenyl]pyrrolo[2,1-f][1,2,4]triazin-6-yl}propanoate; 4-amino-7-cyclopentyl-5-(4-phenoxyphenyl)pyrrolo[2,1-f][1,2,4]triazine-6-carboxylic acid; 3-{4-amino-5-[4-(3-chlorophenoxy)-3-methoxyphenyl]-7-(1-propen-2-yl)pyrrolo[2,1-f][1,2,4]triazin-6-yl)propanoic acid; (2E)-3-[4-amino-7-cyclopentyl-5-(4 phenoxyphenyl)pyrrolo[2,1-f][1,2,4]triazin-6-yl]acrylonitrile; methyl({[4-amino-7-cyclopentyl-5-(4-phenoxyphenyl)pyrrolo[2,1-f][1,2,4]triazin-6-yl]carbonyl}amino)acetate; 6-fluoro-7-(3-methoxyphenyl)-5-(4-phenoxyphenyl)pyrrolo[2,1-f][1,2,4]tria-zin-4-amine; ethyl(2E)-3-[7-(1-acryoyl-3-methoxy-3-azetidinyl)-4-amino-5-(4-phenoxyphenyl)-pyrrolo[2,1-f][1,2,4]trazin-6-yl]acrylate; 3-[4-amino-7-cyclopentyl-5-(4-phenoxyphenyl)pyrrolo[2,1-f][1,2,4]triazin-6-yl]-2-methylacrylic acid; (2E)-3-{4-amino-7-cyclopentyl-5-[4-(3,4-dichlorophenoxy)-3-methoxyphenyl]-pyrrolo[2,1-f][1,2,4]triazin-6-yl}acrylic acid; (2E)-3-[4-amino-7-cyclopentyl-5-(4-phenoxyphenyl)pyrrolo[2,1-f][1,2,4]triazin-6-yl]acrylamide; (2E)-3-[4-amino-7-cyclopentyl-5-(4-phenoxyphenyl)pyrrolo[2,1-f][1,2,4]triazin-6-yl]-2-propen-1-ol; 6-(2-aminoethyl)-7-cyclopentyl-5-(4-phenoxyphenyl)pyrrolo[2,1-f][1,2,4]triazin-4-amine; (2E)-3-[4-amino-7-cyclopentyl-5-(4-phenoxyphenyl)pyrrolo[2,1-f][1,2,4]triazin-6-yl]acrylohydrazide; 3-{4-amino-5-[4-(3,4-dichlorophenoxy)-3-methoxyphenyl]-7-(3-methoxyphenyl)pyrrolo[2,1-f][1,2,4]triazin-6-yl}propanamide; (2E)-1-(3-{4-amino-6-chloro-5-[4-(3,4-dichlorophenoxy)-3-methoxyphenyl]pyrrolo[2,1-f][1,2,4]triazin-7-yl}-1-piperidinyl)-4-(dimethylamino)-2-buten-1-one; 1-(3-{4-amino-6-chloro-5-[4-(3-chlorophenoxy)-3-methoxyphenyl]pyrrolo[2,1-f][1,2,4]triazin-7-yl}-1-pyrrolidinyl)-2-propen-1-one; 3-{7-(1-acryloyl-3-piperidinyl)-4-amino-5-[4-(3-chlorophenoxy)-3-methoxyphenyl]pyrrolo[2,1-f][1,2,4]triazin-6-yl}propanoic acid; 3-{4-amino-5-[4-(3-chlorophenoxy)-3-methoxyphenyl]-7-cyclopentylpyrrolo[2,1-f][1,2,4]triazin-6-yl}propanoic acid, 4-{4-amino-5-[4-(3-chlorophenoxy)-3-methoxyphenyl]-7-(3-methoxyphenyl)pyrrolo[2,1-f][1,2,4]triazin-6-yl}butanoic acid; 4-{4-amino-5-[4-(3,4-dichlorophenoxy)-3-methoxyphenyl]-7-(3-methoxyphenyl)pyrrolo[2,1-f][1,2,4]triazin-6-yl}butanoic acid; 3-(4-amino-5-[4-(3-chlorophenoxy)-3-methoxyphenyl]-7-{4-[(methylsulfonyl)-amino]phenyl)pyrrolo[2,1-f][1,2,4]triazin-6-yl}propanoic acid; (2E)-3-[4-amino-7-(3-methoxyphenyl)-5-(4-phenoxyphenyl)pyrrolo[2,1-f][1,2,4]triazin-6-yl]acrylic acid; 1-(3-{4-amino-6-chloro-5-[4-(3-chlorophenoxy)-3-methoxyphenyl]pyrrolo[2,1-f][1,2,4]triazin-7-yl}-3-methoxy-1-azetidinyl)-2-propen-1-one; 5-[4-(3,4-dichlorophenoxy)-3-methoxyphenyl]-7-(3-methoxyphenyl)-6-[2-(1H-tetrazol-5-yl)ethyl]pyrrolo[2,1-f][1,2,4]triazin-4-amine; 3-{4-amino-5-[4-(3-chlorophenoxy)phenyl]-7-cyclopentylpyrrolo[2,1-f][1,2,4]triazin-6-yl}propanoic acid; 3-{4-amino-7-cyclopentyl-5-[4-(3,4-dichlorophenoxy)phenyl]pyrrolo[2,1-f][1,2,4]triazin-6-yl}propanoic acid; 3-{4-amino-7-cyclopentyl-5-[4-(3,4-dichlorophenoxy)-3-methoxyphenyl]pyrrolo[2,1-f][1,2,4]triazin-6-yl}propanoic acid; 3-(4-amino-7-cyclopentyl-5-{4-{3-(2-propanyl)phenoxy}phenyl}pyrrolo[2,1-f][1,2,4]triazin-6-yl)propanoic acid; 3-(7-(1-acryloyl-1,2,5,6-tetrahydro-3-pyridinyl)-4-amino-5-[4-(3-chlorophenoxy)-3-methoxyphenyl]pyrrolo[2,1-f][1,2,4]triazin-6-yl)propanoic acid; 3-[4-amino-7-cyclopentyl-5-(4-phenoxyphenyl)pyrrolo[2,1-f][1,2,4]triazin-6-yl]propanoic acid; 3-[4-amino-7-cyclopentyl-5-(3-hydroxyphenyl)pyrrolo[2,1-f][1,2,4]triazin-6-yl]propanoic acid; 1-(3-{4-amino-6-chloro-5-[4-(3-chlorophenoxy)-3-hydroxyphenyl]pyrrolo[2,1-f][1,2,4]triazin-7-yl}-1-piperidinyl)-2-propen-one; 3-{4-amino-5-[4-(3-chlorophenoxy)-3-methoxyphenyl]-7-(3-methoxyphenyl)pyrrolo[2,1-f][1,2,4]triazin-6-yl}propanoic acid; 3-{4-amino-5-[4-(3-chlorophenoxy)-3-methoxyphenyl]-7-(4-methoxyphenol)pyrrolo[2,1-f][1,2,4]triazin-6-yl}propanoic acid; 3-{7-(1-acryloyl-3-piperdinyl)-4-amino-5-[4-(3,4-dichlorophenoxy)-3-methoxyphenyl]pyrrolo[2,1-f][1,2,4]triazin-6-yl}propanoic acid; 3-{4-amino-5-[4-(3-chlorophenoxy)-3-methoxyphenyl]-7-phenylpyrrolo[2,1-f]-[1,2,4]triazin-6-yl}propanoic acid; 3-{4-amino-5-[4-(3-chlorophenoxy)-3-methoxyphenyl]-7-(2-fluorophenyl)pyrrolo[2,1-f][1,2,4]triazin-6-yl}propanoic acid; 3-{4-amino-5-[4-(3-chlorophenoxy)-3-methoxyphenyl]-7 (3-fluorophenyl)pyrrolo[2,1-f][1,2,4]triazin-6-yl}propanoic acid; 3-{4-amino-5-[4-(3chlorophenoxy)-3-methoxyphenyl]-7-(4-fluorophenyl)pyrrolo[2,1-f][1,2,4]triazin-6-yl}propanoic acid; 3-{4-amino-7-cyclopentyl-5-[4-(3,5-difluorophenoxy)-3-methoxyphenyl]pyrrolo[2,1-f][1,2,4]triazin-6-yl}propanoic acid; 3-{4-amino-7-cyclopentyl-5-[4-(3,5-dichlorophenoxy)-3-methoxyphenyl]pyrrolo[2,1-f][1,2,4]triazin-6-yl}propanoic acid; 3-{4-amino-7-(3-carbamoylphenyl)-5-[4-(3-chlorophenoxy)-3-methoxyphenyl]pyrrolo[2,1-f][1,2,4]triazin-6-yl}propanoic acid; 3-{4-amino-7-cyclopentyl-5-[4-(3,5-difluorophenoxy)phenyl]pyrrolo[2,1-f][1,2,4]trazin-6-yl}propanoic acid; 3-{4-amino-7-cyclopentyl-5-[4-(3,5-dichlorophenoxy)phenyl]pyrrolo[2,1-f][1,2,4]triazin-6-yl}propanoic acid; (2E)-3-[4-amino-7-cyclopentyl-5-(4-phenoxyphenyl)pyrrolo[2,1-f][1,2,4]triazin-6 yl]acrylic acid; 3-{4-amino-S-[4-(3-chlorophenoxy)-3-methoxyphenyl]-7-(2-hydroxy-2-propanyl)pyrrolo[2,1-f][1,2,4]triazin-6-yl}propanoic acid; ({[4-amino-7-cyclopentyl-5-(4-phenoxyphenyl)pyrrolo[2,1-f][1,2,4]triazin-6-yl]carbonyl)amino)acetic acid; 3-(4-amino-5-[4-(3-chlorophenoxy)-3-methoxyphenyl]-7-{4-[methyl(methylsulfonyl)amino]phenyl})pyrrolo[2,1-f][1,2,4]triazin-6-yl)propanoic acid; 3-{4-amino-5-[4-(3-chlorophenoxy)-3-methoxyphenyl]-7-[4-(methylsulfonyl)phenyl]pyrrolo[2,1-f][1,2,4]triazin-6-yl}propanoic acid; 1-[5-{4-amino-6-chloro-5-[4-(3-chlorophenoxy)-3-methoxyphenyl]pyrrolo[2,1-f][1,2,4]triazin-7-yl}-3,6-dihydro-1-(2H)-pyridinyl]-2-propen-1-one; 3-[4-amino-7-{-4-[methyl(methylsulfonyl)amino]phenyl}-5-{4-phenoxyphenyl)pyrrolo[2,1-f][1,2,4]triazin-6-yl]propanoic acid; (2E)-3-[4-amino-7-(2-hydroxy-2-propanyl)-5-(4-phenoxyphenyl)pyrrolo[2,1-f][1,2,4]triazin-6-yl]acrylic acid; 3-(4-amino-7-cyclopentyl-5-{3-methoxy-4-[3-(2-propanyl)phenoxy]phenyl})pyrrolo[2,1-f][1,2,4]triazin-6-yl}propanoic acid; (2E)-3-[7-(1-acryloyl-3-methoxy-3-azetidinyl)-4-amino-5-(4-phenoxyphenyl)-pyrrolo[2,1-f][1,2,4]triazin-6-yl]acrylic acid; (2E)-3-{4-amino-7-[1-(methylsulfonyl)-4-piperidinyl]-5-(4-phenoxyphenyl)pyrrolo[2,1-f][1,2,4]triazin-6-yl}acrylic acid; (2E)-3-[4-amino-7-cyclopentyl-5-{2-fluoro-4-phenoxyphenyl)pyrrolo[2,1-f][-1,2,4]triazin-6-yl]acrylic acid; 3-[4-amino-7-(3-methoxyphenyl)-5-(4-phenoxyphenyl)pyrrolo[2,1-f][1,2,4]triazin-6-yl]propanoic acid; (2E)-3-[4-amino-7-cyclohexyl-5-(4-phenoxyphenyl)pyrrolo[2,1-f][1,2,4]triazin-6-yl]acrylic acid; (2E)-3-[4-amino-7-(1,1-dioxidotetrahydro-2H-thiopyran-4-yl}-5-(4-phenoxyphenyl)pyrrolo[2,1-f][1,2,4]triazin-6-yl]acrylic acid, (2E)-3-(4-amino-7-cyclopentyl-5-{4-[3-(2-propanyl)phenoxy]phenyl}pyrrolo[2,1-f][1,2,4]triazin-6-yl)acrylic acid; (2E)-3-{4-amino-5-[4-(3-chlorophenoxy)-3-methoxyphenyl]-7-cyclopentylpyrrolo[2,1-f][1,2,4]triazin-6-yl}acrylic acid; (2E)-3-[4-amino-5-(4-phenoxyphenyl)-7-(tetrahydro-2H-pyran-4-yl))pyrrolo[2,1-f][1,2,4]triazin-6-yl]acrylic acid; (2E)-3-[4-amino-7-(3-hydroxy-3-methylbutyl)-5-(4-phenoxyphenyl)pyrrolo[2,-1-f][1,2,4]triazin-6-yl]acrylic acid; (2E)-3-[4-amino-7-isopropyl-5-(4-phenoxyphenyl)pyrrolo[2,1-f][1,2,4]triazin-6-yl]acrylic acid; N-{2-(4-amino-7-cyclopentyl-5-(4-phenoxyphenyl)pyrrolo[2,1-f][1,2,4]triazin-6-yl]ethyl}acetamide; 1-[4-amino-7-cyclopentyl-5-(4-phenoxyphenyl)pyrrolo[2,1-f][1,2,4]triazin-6-yl]-1,2-ethanediol; 4-[4-amino-7-cyclopentyl-5-(4-phenoxyphenyl)pyrrolo[2,1-f][1,2,4]triazin-6-yl]-1-hydroxy-2-butanone; 4-amino-7-cyclopentyl-N-(2-hydroxyethyl)-5-(4-phenoxyphenyl)pyrrolo[2,1-f][1,2,4]triazine-6-carboxamide; 4-amino-7-cyclopentyl-5-(4-phenoxyphenyl)-N-(1H-pyrazol-4-yl)pyrrolo[2,1-f][1,2,4]triazine-6-carboxamide; 4-amino-7-cyclopentyl-5-(4-phenoxyphenyl)-N-(1H-pyrazol-3-yl)pyrrolo[2,1-f][1,2,4]triazine-6-carboxamide; (2E)-3-[4-amino-7-cyclopentyl-5-(4-phenoxyphenyl)pyrrolo[2,1-f][1,2,4]triazin-6-yl]acrylamide; 4-amino-7-cyclopentyl-N-(2-methoxyethyl)-5-(4-phenoxyphenyl)pyrrolo[2,1-f][1,2,4]triazine-6-carboxamide; 4-amino-7-cyclopentyl-S-(4 phenoxyphenyl)pyrrolo[2,1-f][1,2,4]triazine-6-carbohydrazide; 4-amino-7-cyclopentyl-N-[2-(dimethylamino)ethyl]-5-(4-phenoxyphenyl)pyrrolo[2,1-f][1,2,4]triazine-6-carboxamide; (2E)-3-[4-amino-7-cyclopentyl-5-(4-phenoxyphenyl)pyrrolo[2,1-f][1,2,4]triazin-6-yl]-N-(1H-pyrazol-4-yl)acrylamide; and 4-amino-7-cyclopentyl-5-(4-phenoxyphenyl)pyrrolo[2,1-f][1,2,4]triazine-6-carboxamide.

Particular compounds of Formula III include, but are not limited to, 1-(3-(4-amino-3-(4-phenoxyphenyl)-1H-pyrazolo[3,4-d]pyrimidin-1-yl)piperidin-1-yl)prop-2-en-1-one; (E)-1-(3-(4-amino-3-(4-phenoxyphenyl)-1H-pyrazolo[3,4d]pyrimidin-1-yl)piperidin-1-yl)but-2-en-1-one; 1-(3-(4-amino-3-(4-phenoxyphenyl)-1H-pyrazolo[3,4d]pyrimidin-1-yl)piperidin-1-yl)sulfonylethene; 1-(3-(4-amino-3-(4-phenoxyphenyl)-1H-pyrazolo[3,4-d]pyrimidin-1-yl)piperidin-yl)prop-2-yn-1-one; 1-(4-(4-amino-3-(4-phenoxyphenyl)-1H-pyrazolo[3,4d]pyrimidin-1-yl)piperidin-1-yl)prop-2-yn-1-one; N-((1s,4s)-4-(4-amino-3-(4-phenoxyphenyl)-1H-pyrazolo[3,4d]pyrimidin-1-yl)cyclohexyl)acrylamide; 1-((R)-3-(4-amino-3-(4-phenoxyphenyl)-1H-pyrazolo[3,4-d]pyrimidin-1-yl)pyrrolidin-1-yl)prop-2-en-1-one; 1-((S)-3-(4-amino-3-(4-phenoxyphenyl)-1H-pyrazolo[3,4d]pyrimidin-1-yl)pyrrolidin-1-yl)prop-2-en-1-one; 1-((R)-3-(4-amino-3-(4-phenoxyphenyl)-1H-pyrazolo[3,4-d]pyrimidin-1-yl)piperidin-1-yl)prop-2-en-1-one; 1-((S)-3-(4-amino-3-(4-phenoxyphenyl)-1H-pyrazolo[3,4-d]pyrimidin-1-yl)piperidin-1-yl)prop-2-en-1-one; and (E)-1-(3-(4-amino-3-(4-phenoxyphenyl)-1H-pyrazolo[3,4-d]pyrimidin-1-yl)piperidin-1-yl)-4-(dimethylamino)but-2-en-1-one.

As used herein, the term “irreversibe” or “irreversible inhibitor” refers to an inhibitor (i.e., a compound) that is able to be covalently bonded to a target protein kinase in a substantially non-reversible manner. That is, whereas a reversible inhibitor is able to bind to (but is generally unable to form a covalent bond) the target protein kinase, and therefore can become dissociated from the target protein kinase, an irreversible inhibitor will remain substantially bound to the target protein kinase once covalent bond formation has occurred. Irreversible inhibitors usually display time dependency, whereby the degree of inhibition increases with the time with which the inhibitor is in contact with the enzyme. Methods for identifying if a compound is acting as an irreversible inhibitor are known to one of ordinary skill in the art. Such methods include, but are not limited to, enzyme kinetic analysis of the inhibition profile of the compound with the protein kinase target, the use of mass spectrometry of the protein drug target modified in the presence of the inhibitor compound, discontinuous exposure, also known as “washout,” experiments, and the use of labeling, such as radiolabelled inhibitor, to show covalent modification of the enzyme, as well as other methods known to one of skill in the art.

One of ordinary skill in the art will recognize that certain reactive functional groups can act as “warheads.” As used herein, the term “warhead” or “warhead group” refers to a functional group present on a compound of the present invention wherein that functional group is capable of covalently binding to an amino acid residue (such as cysteine, lysine, histidine, or other residues capable of being covalently modified) present in the binding pocket of the target protein, thereby irreversibly inhibiting the protein. It will be appreciated that the -L-Y group, as defined and described herein, provides such warhead groups for covalently, and Irreversibly, inhibiting the protein.

As used herein, the term “inhibitor” is defined as a compound that binds to and/or inhibits the target protein kinase with measurable affinity. In certain embodiments, an inhibitor has an IC₅₀ and/or binding constant of less about 50 μM, less than about 1 μM, less than about 500 nM, less than about 100 nM, or less than about 10 nM.

As defined generally above, R¹ is a warhead group, or, when R¹ and R² form a ring, then -Q-Z is a warhead group. Without wishing to be bound by any particular theory, it is believed that such R¹ groups, i.e., warhead groups, are particularly suitable for covalently binding to a key cysteine residue in the binding domain of certain protein kinases.

In some embodiments, provided compounds are irreversible inhibitors of a TEC-kinase (e.g. BTK). One of ordinary skill in the art will recognize that certain compounds of the present invention are reversible inhibitors. In certain embodiments, such compounds are useful as assay comparator compounds. In other embodiments, such reversible compounds are useful as inhibitors of a TEC-kinase or a mutant thereof, and therefore useful for treating one or disorders as described herein.

### Methods, Formulation and Administration

The invention provides a method of treating a skeletal related disorder comprising administering a composition comprising a compound described herein or a pharmaceutically acceptable derivative thereof and a pharmaceutically acceptable carrier or vehicle. The amount of compound in compositions is such that is effective to measurably inhibit a Tec protein kinase, including BTK, in a subject. In certain embodiments, the skeletal disorder is a bone fracture or spinal compression resulting from metastatic bone cancer. In certain embodiments, the primary cancer in the subject is lung cancer, prostate cancer, multiple myeloma or a lymphoma. In some embodiments, the lymphoma is a mature B cell lymphoma.

In some embodiments, the method provides for treating the skeletal related disorder by inhibiting bone resorption in subject, said method comprising administering to the subject a composition comprising a therapeutically effective amount of a compound that is a BTK inhibitor as described herein, or a pharmaceutically acceptable salt thereof. In certain embodiments, methods of Inhibiting or preventing the loss of bone mass and/or for increasing bone formation in a subject who is suffering from metastatic bone cancer. These methods comprise administering to the subject a composition comprising a therapeutically effective amount of a compound described herein, or a pharmaceutically acceptable salt thereof.

In certain embodiments are provided methods for preventing or inhibiting bone deterioration in subjects at risk for loss of bone mass, including human subjects suffering from metastatic bone cancer. Yet another object is to provide methods for repairing defects in the micro structure of structurally compromised bone, including repairing bone fractures in such human subjects.

In some embodiments are provided methods and compositions for stimulating bone formation and increasing bone mass, optionally over prolonged periods of time, and particularly to decrease the occurrence of or prevent new fractures resulting from structural deterioration of the skeleton.

In certain embodiments are methods of treating a skeletal-related condition such as a bone fracture or spinal compression in a subject suffering from metastatic bone cancer, said methods comprising administering to a cancer subject in need thereof, a compound described herein which is a BTK inhibitor or a pharmaceutically acceptable salt thereof, wherein said treatment results in preservation of bone density. In certain embodiments, the cancer is a relapsed or refractory cancer. In other embodiments, the cancer is a newly diagnosed cancer. In certain embodiments, the cancer is multiple myeloma. In certain embodiments, the cancer is a relapsed or refractory multiple myeloma. In some embodiments, the cancer is a newly diagnosed multiple myeloma. In some embodiments, the multiple myeloma is a stage 1 multiple myeloma. In other embodiments, the multiple myeloma is a stage 2 multiple myeloma. In other embodiments, the multiple myeloma is a stage 3 multiple myeloma. In other embodiments, the multiple myeloma is a high-risk multiple myeloma. In other embodiments, the multiple myeloma is a treatment naive multiple myeloma. In other embodiments, the multiple myeloma is a recurrent multiple myeloma.

In certain embodiments are methods of treating a skeletal-related condition such as a bone fracture or spinal compression in a subject suffering from metastatic bone cancer, said methods comprising administering to a cancer subject in need thereof, a compound described herein which is a BTK inhibitor or a pharmaceutically acceptable salt thereof, wherein said treatment results in preservation of bone density. In certain embodiments, the cancer is a relapsed or refractory cancer. In other embodiments, the cancer is a newly diagnosed cancer. In certain embodiments, the cancer is a blood cancer such as lymphoma. In certain embodiments, the cancer is a relapsed or refractory lymphoma. In some embodiments, the cancer is a newly diagnosed lymphoma. In some embodiments, the lymphoma is selected from Hodgkin's lymphoma or non-Hodgkin's lymphoma. In other embodiments, the lymphoma is a mature B cell neoplasm including chronic lymphocytic leukemia, small lymphocytic lymphoma, B-cell prolymphocytic leukemia, lymphoplasmacytic lymphoma (such as Waldenström macroglobulinemia), Splenic marginal zone lymphoma, plasma cell neoplasms including plasma cell myeloma & plasmacytoma, follicular lymphoma, mantle cell lymphoma, diffuse large B cell lymphoma, mediastinal (thymic) large B cell lymphoma, intravascular large B cell lymphoma, primary effusion lymphoma and Burkitt lymphoma/leukemia.

In some embodiments, the subject is suffering from metastatic bone cancer wherein the primary cancer is selected from the group consisting of breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, melanoma, Ewing's sarcoma, reticulum cell sarcoma, myeloma, giant cell tumor, small-cell lung tumor, islet cell tumor, primary brain tumor, acute and chronic lymphocytic and granulocytic tumors, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, and epidermoid carcinomas. In other embodiments, the cancer being treated is pancreatic cancer, liver cancer, breast cancer, osteosarcoma, lung cancer, soft tissue sarcoma, cancer of the larynx, melanoma, ovarian cancer, brain cancer, Ewing's sarcoma or colon cancer. In other embodiments, the subject suffering from the cancer is elderly.

The term “subject” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this Invention or an inhibitorily active metabolite or residue thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and Intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a subject receiving these compositions.

The compounds and compositions, according to the method of the present invention, may be administered using any amount and any route of administration effective for treating or lessening the severity a skeletal-related disorder, including bone fractures and spinal compression. The exact amount required will vary from subject to subject, depending on the species, age, and general condition of the subject, the severity of the infection, the particular agent, its mode of administration, and the like. Compounds described herein are formulated in dosage unit form for ease of administration and uniformity of dosage. The expression “dosage unit form” as used herein refers to a physically discrete unit of agent appropriate for the subject to be treated. It will be understood, however, that the total daily usage of the compounds and compositions described herein will be decided by the attending physician within the scope of sound medical judgment. The specific effective dose level for any particular subject or organism will depend upon a variety of factors including the severity of the skeletal-related disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed, and like factors well known in the medical arts.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed Including synthetic mono- or diglycerides. In addition, fatty acids such as oleic acid are used in the preparation of injectables.

Injectable formulations can be sterilized, for example, by filtration through a bacterial-retaining filter, or by Incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, hi absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Another embodiment of the present invention relates to a method of inhibiting BTK activity in a subject comprising the step of administering to said subject a compound of the present invention, or a composition comprising said compound.

According to another embodiment, the invention relates to a method of inhibiting BTK and/or a TEC-kinase activity in a subject comprising the step of administering to said subject a compound described herein, or a composition comprising said compound. According to certain embodiments, the invention relates to a method of irreversibly inhibiting BTK and/or a Tec kinase in a subject comprising the step of administering to said subject a compound of the present invention, or a composition comprising said compound. In other embodiments, the present invention provides a method for treating a skeletal-related disorder mediated by BTK or a TEC-kinase in a subject in need thereof, comprising the step of administering to said subject a compound according to the present invention or pharmaceutically acceptable composition thereof. Such disorders are described in detail herein.

Depending upon the particular condition, or disease, to be treated, additional therapeutic agents, which are normally administered to treat that condition, may also be present in the compositions of this invention. As used herein, additional therapeutic agents that are normally administered to treat a particular disease, or condition, are known as “appropriate for the disease, or condition, being treated.”

For example, compounds of the present invention, or a pharmaceutically acceptable composition thereof, are administered in combination with chemotherapeutic agents to treat cancer. Examples of known chemotherapeutic agents include, but are not limited to, Adriamycin, dexamethasone, vincristine, cyclophosphamide, fluorouracil, topotecan, taxol, interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, camptothecin, cisplatin, metronidazole, and Gleevec™, among others. In other embodiments, a compound of the present invention is administered in combination with a biologic agent, such as Avastin or VECTIBIX.

In certain embodiments, compounds described herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostne, anastrozole, arsenic trioxide, asparaginase, azacitidine, BCG Live, bevacuzimab, fluorouracil, bexarotene, bleomycin, bortezomib, busultan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukln, dexrazoxane, docetaxel, doxorubicin (neutral), doxorubicin hydrochloride, dromostanolone propionate, epirubicin, epoetin aifa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, soralenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a provided compound, an additional therapeutic agent, and a pharmaceutically acceptable or vehicle.

The amount of both, a compound described herein and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions described herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Examples of kinases that are inhibited by the compounds and compositions described herein and which the methods described herein are useful include BTK and other TEC-kinases including but not limited to, ITK, TEC, BMX and RLK.

Compounds useful in the methods of the invention and described herein are inhibitors of one of more TEC-kinases and are therefore useful for treating one or more disorders associated with activity of one or more TEC-kinases. Thus, in certain embodiments, the present invention provides a method for treating a TEC-mediated skeletal-disorder comprising the step of administering to a subject in need thereof a compound of the present invention, or pharmaceutically acceptable composition thereof.

The term “TEC-mediated condition” as used herein means any skeletal-related condition in which TEC-kinases are known to play a role. Accordingly, another embodiment of the present invention relates to treating or lessening the severity of one or more diseases in which TEC-kinases are known to play a role. Specifically, the present invention relates to a method of treating or lessening the severity of a skeletal-related disease or condition involving bone resorption and selected from skeletal fractures and spinal compression, wherein said method comprises administering to a subject in need thereof a compound described herein.

In some embodiments, the present invention provides a method for treating or lessening the severity of one or more skeletal-related diseases and conditions associated with TEC-kinases including diseases including bone fractures and spinal compression by reducing bone resorption in the subject.

In some embodiments, the present invention provides a method for treating or lessening the severity of one or more skeletal-related diseases and conditions associated with TEC-kinases including diseases of the bone in subjects suffering from bone cancer, including metastatic bone cancer.

Bruton's tyrosine kinase (“BTK”), a member of the TEC-kinase family, is a key signaling enzyme expressed in all hematopoietic cell types except T lymphocytes and natural killer cells. BTK plays an essential role in the B-cell signaling pathway linking cell surface B-cell receptor (BCR) stimulation to downstream intracellular responses.

Provided compounds are inhibitors of BTK and are therefore useful for treating one or more skeletal-related disorders associated with activity of BTK. Thus, in some embodiments, the present invention provides a method for treating a BTK-mediated disorder comprising the step of administering to a subject in need thereof a compound of the present invention, or pharmaceutically acceptable composition thereof.

As used herein, the term “BTK-mediated” skeletal disorders or conditions as used herein means any disease or other deleterious condition in which BTK, or a mutant thereof, is known to play a role. Accordingly, another embodiment of the present invention relates to treating or lessening the severity of one or more diseases in which BTK, or a mutant thereof, is known to play a role. Specifically, the present invention relates to a method of treating or lessening the severity of a disease or condition related to bone resorption, wherein said method comprises administering to a subject in need thereof a compound or composition according to the present invention.

One advantage of the methods of the invention is treatment of patients who undergo dental procedures while receiving treatment of skeletal disorders described herein. For example, patients who receive Denosumab or bisphosphonate therapy for a skeletal disorder often develop a rare side effect known as osteonecrosis of the jaw due to the inhibition of RANKL by Denosumab and other bisphosphonates. This side effect has been attributed to the long half-life of Denosumab and other bisphosphonates which remain in the blood stream and continue to inhibit RANKL activity, resulting in the osteonecrosis. The compounds described herein all have a shorter half-life when compared to Denosumab and bisphosphonates and as such, will not produce osteonecrosis of the jaw in patients who undergo dental procedures given their advantageous pharmacokinetic profile when compared to current therapies.

Without further description, it is believed that one of ordinary skill in the art can, using the preceding description and the following illustrative examples, make and utilize the present invention and practice the claimed methods. The following working examples therefore, specifically point out some embodiments of the present invention, and are not to be construed as limiting in any way the remainder of the disclosure.

### EXAMPLES

### Example 1

### Effect of Inhibitors on Bone Density in Bone Metastasis Model

Nude (rnu/rnu) female rats were obtained from Charles River (Wilmington, Mass.). The rats were pair-housed in polycarbonate micro-isolator cages lined with autoclaved bedding. Autoclaved reverse osmosis (RO) water and autoclaved standard rat chow were provided ad libitum. Body weights were recorded weekly throughout the course of the studies. Animals were maintained for 6 weeks and euthanized by carbon dioxide inhalation at the end of the experiment. The animals were randomized to study groups by body weight as follows: G1: vehicle BID PO (N=6), G2: 0.04 mg/kg zoledronate QWK IV (N=6), G5: 90 mg/kg Compound of Formula IV BID PO (N=6), and G6: No implantation (N=3).

MDA-MB-231 breast carcinoma cells were propagated by standard tissue culture methods in RPMI media containing 10% fetal bovine serum. Exponentially growing cells were trypsinized and resuspended in phosphate-buffered saline at a concentration of 2.5×10⁷ cells/mi. Rats were maintained under isoflurane anesthesia during the tumor injection procedure. The skin surface at the injection site was aseptically prepared with betadine scrub followed by an alcohol wipe. A 23-gauge needle with a 1-mL syringe was inserted extracapsullarly through the tibial crest, epiphysis, and growth plate. Five million tumor cells in a 0.2 ml volume were injected into the bone marrow space of the right tibial metaphysis. Rats with radiographically detectable tumors at 2 weeks after injection were selected for inclusion in the study.

Radiographs were taken with rats under general anaesthesia. Animals were placed in a.p. position on a high-resolution mammography film and exposed to an X-ray at 55 kV, 5 mA for 90 sec using a radiographic inspection unit for animals. Radiographs were scanned and analysed. Metastatic foci |0.5 mm in diameter were recognised as radiolucent lesions and manually delineated to determine the number (n), the perimeter (mm) and the area (mm²) of osteolytic lesions. Since Initially separated lesions showed a strong tendency to confluate during tumour growth, only the total osteolytic area per bone and animal was used for further analyses.

Femoral and tibial X-ray densitometry was performed. In brief, bones were X-rayed, the film was scanned and the electronic image then analysed by a real-colour image analysis system. Pixel density was then determined and used as a measure of disease progression.

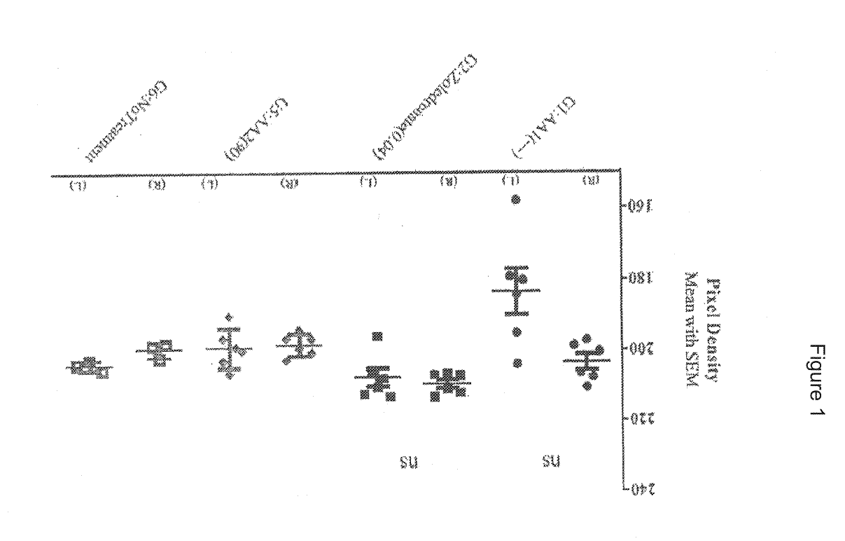

Results are presented in FIG. 1 and demonstrate inhibition of the decrease in bone density following administration of the Compound of Formula IV (see Group G5).

Although the present invention has been described in detail with reference to examples above, it is understood that various modifications can be made without departing from the spirit of the invention. Accordingly, the invention is limited only by the following claims. All patents, published patent applications, and other published references cited herein are incorporated by reference in their entirety.

1. A method of preventing and/or treating a skeletal-related event in a human subject with bone metastases from solid tumors comprising administering to the human subject an amount of a compound of Formula I-a or I-b: 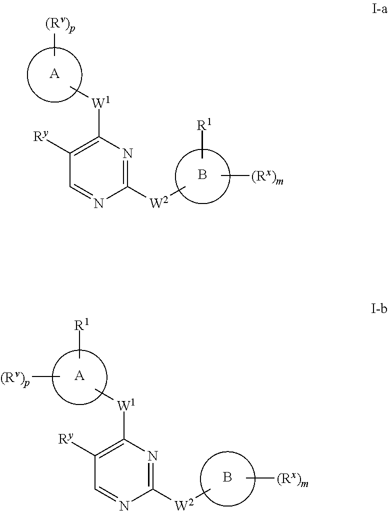
or a pharmaceutically acceptable salt thereof,
effective to inhibit the activity of Bruton's tyrosine kinase (Btk) in the human subject, wherein:
Ring A is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
Ring B is an optionally substituted group selected from phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, or sulfur, a 4-7 membered saturated or partially unsaturated heterocyclic ring having 1-3 heteroatoms independently selected from nitrogen, oxygen, or sulfur, a 7-10 membered bicyclic saturated or partially unsaturated heterocyclic ring having 1-5 heteroatoms independently selected from nitrogen, oxygen, or sulfur, or an 8-10 membered bicyclic heteroaryl ring having 1-5 heteroatoms independently selected from nitrogen, oxygen, or sulfur;
R¹ is a warhead group;
R^{y} is hydrogen, halogen, —CN, —CF₃, C₁₋₄ aliphatic, C₁₋₄ haloaliphatic, —OR, —C(O)R, or —C(O)N(R)₂;
each R group is independently hydrogen or an optionally substituted group selected from C₁₋₆ aliphatic, phenyl, a 4-7-membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
W¹ and W² are each independently a covalent bond or a bivalent C₁₋₃ alkylene chain wherein one methylene unit of W¹ or W² is optionally replaced by —NR₂—, —N(R²)C(O)—, —C(O)N(R²)—, —N(R²)SO₂—, —SO₂N(R²)—, —O—, —C(O)—, —OC(O)—, —C(O)O—, —S—, —SO— or —SO₂—;
R² is hydrogen, optionally substituted C₁₋₆ aliphatic, or —C(O)R, or:
R² and a substituent on Ring A are taken together with their intervening atoms to form a 4-6 membered partially unsaturated or aromatic fused ring; or
R² and R^{y} are taken together with their intervening atoms to form a 4-6 membered saturated, partially unsaturated, or aromatic fused ring; m and p are independently 0-4; and
Rₓ and R^{v} are independently selected from —R, halogen, —OR, —O(CH₂)_{q}OR, —CN, —NO₂, —SO₂R, —SO₂N(R)2, —SOR, —C(O)R, —CO₂R, —C(O)N(R)₂, —NRC(O)R, —NRC(O)N(R)₂, —NRSO₂R, or —N(R)₂, wherein q is 1-4; or:
R^{x} and R¹ when concurrently present on Ring B are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, CN, or C₁₋₆ aliphatic; or
R^{v} and R¹ when concurrently present on Ring A are taken together with their intervening atoms to form a 5-7 membered saturated, partially unsaturated, or aryl ring having 0-3 heteroatoms independently selected from nitrogen, oxygen, or sulfur, wherein said ring is substituted with a warhead group and 0-3 groups independently selected from oxo, halogen, CN, or C₁₋₆ aliphatic.

2. A method of preventing and/or treating a skeletal-related event in a human subject with bone metastases from a cancer comprising administering to the human subject an amount of a compound of Formula II 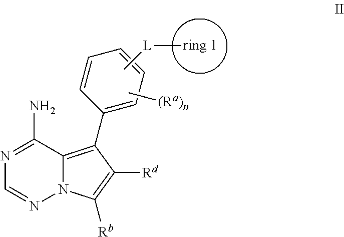
or a pharmaceutically acceptable salt thereof,
effective to inhibit the activity of Bruton's tyrosine kinase (Btk) in the human subject, wherein:
ring1 represents (1) a C₅₋₇carbocyclic ring or (2) a 5-10 membered heterocyclic ring, any of which is optionally substituted with 1-5 substituent(s) selected from the group consisting of halogen, a C₁₋₄alkyl, CF₃, nitrile, CONH₂, and OR^{e-103};
R^{a} represents halogen, a C₁₋₄alkyl, or a C₁₋₄alkoxy;
L represents —O—, —SO—, —SO₂—, —NH—, or —C(O)—;
R^{b} represents (1) a C₁₋₄alkyl substituted with OR^{e-103}, (2) C₂₋₄alkenyl, or (3) ring2 optionally substituted with one or more —K—R^{c};
ring2 represents (1) a C₄₋₇ carbocyclic ring or (2) a 4-7 membered heterocyclic ring, any atom of which is optionally substituted with one or more oxo group; K represents bond, a C₁₋₄alkylene, —C(O)CH₂—, —C(O)CH₂CH₂—, —C(O)O—, —CH₂C(O)—, —CH₂C(O)O—, —C(O)—, —CH₂O—, —CH₂CH₂O—, —O—, —OCH₂—, —OCH₂C(O)— or —SO₂—, wherein the left bond binds to ring2;
R^{c} represents (1) hydrogen, (2) NR^{c-101}R^{c-102}, (3) a C₁₋₄alkyl optionally substituted with NR^{c-101}R^{c-102}, (4) a C₂₋₄ alkenyl optionally substituted with NR^{c-101}R^{c-102}, (5) CF₃, (6) nitrile, (7) halogen, or (8) a cyclic ring optionally substituted with 1-5 substituent(s) selected from the group consisting of halogen, a C₁₋₄alkyl, a C₁₋₄alkoxy, CF₃, nitrile and oxo, wherein the cyclic ring is selected from the group consisting of morpholine, pyrrolidine, benzene, piperazine, tetrahydropyran, piperidine, tetrahydrofuran, oxazole, thiazole, pyrazole and oxadiazole;
R^{d} represents (1) halogen, (2) CONR^{d-101}R^{d-102}, (3) CO₂R^{d-103}, (4) ring3, (5) a C₁₋₄CONR^{d-101}R^{d-102}, CO₂R^{d-103}, COR^{d-103}, OR^{d-103}, SOR^{d-103} and SO₂R^{d-103}, or (6) a C₂₋₄alkenyl which is substituted with 1-5 substituent(s) selected from ring4, nitrile, NR^{d-101}R^{d-102}, CONR^{d-101}R^{d-102}, CO₂R^{d-103}, COR^{d-103}, OR^{d-103}, SOR^{d-103} and SO₂R^{d-103};
R^{c-101} and R^{c-102} each independently represent (1) hydrogen, (2) a C₁₋₄alkyl, (3) COR^{c-103}, (4) CONR^{c-103}R^{c-104} or (5) SO₂R^{c-103}, wherein R^{c-103} and R^{c-104} each independently represent hydrogen or a C₁₋₄alkyl;
R^{d-101}, R^{d-102} and R^{d-103} each independently represent (1) hydrogen, (2) COR^{e-103}, (3) NR^{e-101}R^{e-102}, (4) ring5, or (6) a C₁₋₄alkyl optionally substituted with CO₂R^{e-103}, OR^{e-103}, or NR^{e-101}R^{e-102}; R^{e-101},
R^{e-102} and R^{e-103} each independently represent hydrogen or a C₁₋₄alkyl; ring3, ring4 and ring5 each independently represent a 4-7 membered heterocyclic ring optionally substituted with 1-5 substituent(s) selected from the group consisting of halogen, oxo, a C₁₋₄alkyl, a C₁₋₄alkoxy, CF₃, CONR^{e-101}R^{e-102}, CO₂R^{e-103}, SOR^{e-103}, SO₂R^{e-103} and nitrile;
n represents 0, or an integer of 1-4, wherein when n is more than 1, and
each R¹ may be same or different.

3. A method of preventing and/or treating a skeletal-related event in a human subject with bone metastases from solid tumors comprising administering to the human subject an amount of a compound of Formula III 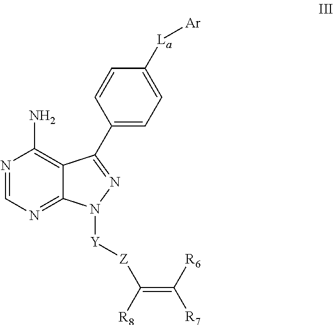
or a pharmaceutically acceptable salt thereof,
effective to inhibit the activity of Bruton's tyrosine kinase (BTK) in the human subject, wherein:
Lₐ is O or S;
Ar is an unsubstituted phenyl;
Y is a 4-, 5-, 6-, or 7-membered cycloalkyl ring, or Y is azetidinyl, pyrrolidinyl, piperidinyl, or azepanyl;
Z is C(═O), OC(═O), NHC(═O), S(═O), or NHS(═O), where x is 2;
R₈ is H;
R₇ is H, unsubstituted C₁₋₄alkyl, C₁₋₆alkoxyalkyl, C₁₋₈alkylaminoalkyl, or C₁₋₄alkyl(phenyl); or
R₇ and R₈ taken together form a bond; and
R₆ is H, unsubstituted C₁₋₄alkyl, C₁₋₆alkoxyalkyl, C₁₋₈alkylaminoalkyl, or C₁₋₄alkyl(phenyl).

4. The method of claim 1 wherein the compound is 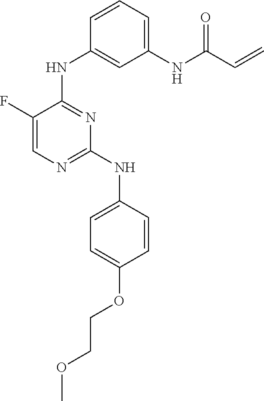 [SMILES: C=CC(=O)Nc1cccc(Nc2nc(Nc3ccc(OCCOC)cc3)ncc2F)c1]
or a pharmaceutically acceptable salt thereof.

5. The method of claim 2 wherein the compound is 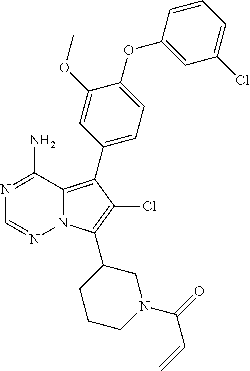 [SMILES: C=CC(=O)N1CCCC(c2c(Cl)c(-c3ccc(Oc4cccc(Cl)c4)c(OC)c3)c3c(N)ncnn23)C1]
or a pharmaceutically acceptable salt thereof.

6. The method of claim 3 wherein the compound is 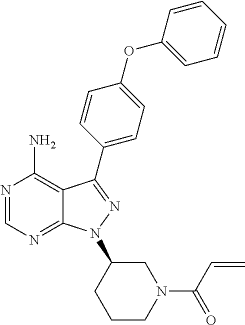 [SMILES: C=CC(=O)N1CCC[C@@H](n2nc(-c3ccc(Oc4ccccc4)cc3)c3c(N)ncnc32)C1]
or a pharmaceutically acceptable salt thereof.

7. The method of claim 1 wherein the skeletal-related event is a bone fracture.

8. The method of claim 1 the skeletal-related event is spinal cord compression.

9. The method of claim 1 wherein the compound of Formula Ia or Ib is administered for treating a subject who has undergone bone surgery to treat a skeletal-related event.

10. The method of claim 2 wherein the compound of Formula II is administered for treating a subject who has undergone bone surgery to treat a skeletal-related event.

11. The method of claim 3 wherein the compound of Formula III is administered for treating a subject who has undergone bone surgery to treat a skeletal-related event.

12. The method of claim 4 wherein the compound is administered for treating a subject who has undergone bone surgery to treat a skeletal-related event.

13. The method of claim 5 wherein the compound is administered for treating a subject who has undergone bone surgery to treat a skeletal-related event.

14. The method of claim 6 wherein the compound is administered for treating a subject who has undergone bone surgery to treat a skeletal-related event.

15. The method of claim 1 wherein the subject is receiving radiation therapy.

16. The method of claim 1 wherein the subject is receiving chemotherapy.

17. The method of claim 1 wherein the subject is suffering from prostate cancer.

18. The method of claim 1 wherein the subject is suffering from multiple myeloma.

19. The method of claim 1 wherein the subject is suffering from breast cancer.

20. The method of claim 1 wherein the subject is suffering from lung cancer.

21. The method of claim 1 wherein Tec tyrosine kinase is also inhibited.

22. The method of claim 1 wherein the compound of Formula Ia or Ib covalently binds to BTK.

23. The method of claim 2 wherein the compound of Formula II covalently binds to BTK.

24. The method of claim 3 wherein the compound of Formula III covalently binds to BTK.

25. The method of claim 1 wherein the compound of Formula Ia or Ib non-covalently binds to BTK.

26. The method of claim 2 wherein the compound of Formula II non-covalently binds to BTK.

27. The method of claim 3 wherein the compound of Formula III non-covalently binds to Btk.

28. The method of claim 1 wherein the compound of Formula Ia or Ib is administered as a pharmaceutical composition.

29. The method of claim 2 wherein the compound of Formula II is administered as a pharmaceutical composition.

30. The method of claim 3 wherein the compound of Formula III is administered as a pharmaceutical composition.

31. The method of claim 4 wherein the compound is administered as a pharmaceutical composition.

32. The method of claim 5 wherein the compound is administered as a pharmaceutical composition.

33. The method of claim 6 wherein the compound is administered as a pharmaceutical composition.

34. The method of claim 28 wherein the pharmaceutical composition is administered orally.
